(19) United States
(12) Patent Application Publication
(10) Pub. No.: US 2006/0183803 A1
(43) Pub. Date: Aug. 17, 2006

(54) L-aspartic acid for the treatment of assorted health problems

(75) Inventors: Enrique Melendez Hevia, Canary Islands (ES); David Melendez Morales, Canary Islands (ES)

Correspondence Address: LARRY J. GUFFEY, WORLD TRADE CENER - SUITE 1800, 401 EAST PRATT STREET, BALTIMORE, MD, 21202, US

(73) Assignee: Instituto del Metabolismo Celular, S.L., Canary Islands (ES)

(21) Appl. No.: 11/356,361

(22) Filed: Feb. 16, 2006

Related U.S. Application Data
(60) Provisional application No. 60/653,704, filed on Feb. 17, 2005.

(51) Int. Cl. A61K 31/198

(52) U.S. Cl. 514/566

(57) ABSTRACT

A method of treating or preventing one's health problem that are associated with one's cells having a poor metabolic capability to process fat includes ingesting a therapeutically effective dosage of an anaplerotic precursor. Examples of such health problems include obesity and the related health conditions that are aggravated by obesity, diabetes, hypertension, atherosclerosis, migraines and headaches, menstrual cramps, cholesterol excesses, multiple sclerosis and Alzheimer's disease, anemia, high blood uric acid levels, ketosis, tobacco and other drug addictions, and digestive problems. In a preferred embodiment, this method includes the daily ingestion of L-aspartic acid, or a pharmaceutically acceptable analog thereof, at the rate of 10-14 g/day and restricting from one's diet the intake of starchy foods.

### CROSS-REFERENCE TO RELATED APPLICATION

This application claims the benefit of U.S. Provisional Patent Application No. 60/653,704, filed Feb. 17, 2005 by David Melendez Morales and Enrique Meléndez Hevia.

### BACKGROUND OF THE INVENTION

1. Field of the Invention

The present invention relates to new therapeutic uses for L-aspartic acid to fight diabetes, obesity, hypertension, blood cholesterol excess and other health problems related with glucose and fat metabolism.

2. Description of the Related Art

Despite significant advancements in biomedicine in recent years, little progress has been seen in the efforts to develop improved treatment methods for a wide range of degenerative diseases that share the characteristic that their victims' bodies exhibit abnormal amounts of fat storage. Examples of diseases within this group include: obesity, diabetes and hypertension.

To better understand the present invention, it proves useful to provide some background information on these and other related diseases:

(a) Obesity

Obesity and overweight are terms that are used to distinguish the conditions of people who have different degrees of excess body mass. These excesses are often quantified in terms a person's body mass index (BMI) which is defined as a person's weight in kilograms (kg) divided by the square of the person's height in meters (m²). (BMI=kg/m²). One having a BMI value in the range of 25-30 is considered to be overweight; a value in the range of 30-35 is considered obese, a value in the range of 35-40 is considered severely obese, and a value higher than 40 is considered very severely or morbidly obese.

Our research indicates that the BMI is not an especially reliable measure of these conditions. A better measure would appear to be one's percentage of body fat. In a research program that we have conducted, we have measured (by Bioclectrical Impedance Analysis methods) the percent of fat of more than 1,500 patients and have seen that it is not uncommon to find people with a high percentage of fat, but a normal BMI (BMI<25).

Endocrinologists, nutritionists and dietitians appear to generally agree that preferred percentages of body fat are: 10-19% in men, and 20-29% in women. Adults who may be at greater risk for cardiovascular diseases have percentages of body fat that are: over 25% in men and over 35% in women.

Many dietitians, nutritionists and endocrinologists insist that the cause and the solution for people's excess percentages of fat are obvious—a consequence of an excess of food intake, mainly fats and sugars. We think, however, that the problem is much more complex, and the aim of our research has been to identify the basic cause of these conditions and to put forth a solution.

(b) Diabetes

Diabetes reportedly has a greater number of adverse health effects upon the world's population than any of the above mentioned health conditions. In 2002, 6.3% of the U.S. population suffered from diabetes (i.e., 0.3% of those under the age of 20 years, 8.7% of those over the age of 20, and 18.3 of those over the age of 60). It is the fifth leading cause of death by disease in the U.S. People with diabetes also are at higher risk for other health problems, including heart disease and stroke, high blood pressure, blindness, kidney disease, nervous system disorders, amputations, dental diseases, complications to pregnancy, biochemical imbalances and susceptibility to other illnesses.

The costs of diabetes are extremely high and are increasing every year. In 2002 in the U.S., these costs were a significant fraction of the total costs for all medical expenditures. Health care spending for people with diabetes is more than double the expenditures for people without diabetes.

Type-1 diabetes (insulin-dependent or juvenile-onset diabetes) accounts for 5% to 10% of the reported cases. It is an autoimmune disease that develops when the body's immune system destroys pancreatic beta cells, which make the hormone insulin. Type-2 diabetes (non-insulin-dependent or adult-onset diabetes) accounts for the other 90% to 95% of the cases. It usually begins as insulin resistance, a disorder in which the cells do not use insulin properly. As the need for insulin rises, the pancreas gradually can lose its ability to produce insulin. Type-2 diabetes currently is being increasingly diagnosed in children and adolescents.

(c) Hypertension

Hypertension (high blood pressure) is a major health problem which results in a significant number of deaths every year. It is often referred to as the “silent killer” since 95% of its cases have an unknown etiology. A review of hypertension can be found in “Kaplan's Clinical Hypertension,” 8th ed. (2002), Kaplan, N. M. & Lieberman, E.

Hypertension is recognized as one of the risk factors that are used to diagnose what is known as the “metabolic syndrome” (the dominant underlying risk factors for this syndrome appear to be abdominal obesity and insulin resistance—a generalized metabolic disorder, in which the body can't use insulin efficiently).

People with the metabolic syndrome are at increased risk of coronary heart disease and other diseases related to plaque buildups in artery walls (e.g., stroke and peripheral vascular disease) and type-2 diabetes. The metabolic syndrome has become increasingly common in the United States. It's estimated that over 50 million Americans have it.

(d) Other Health Problems Associated With One Having Excess Fat Deposits

There are a number of other health problems that have been associated with one's having excess fat deposits. These include: headaches, migraines, menstrual pains, infertility, impotence, heart disease, high cholesterol levels, and even some forms of cancer, especially breast and colon, and gallbladder disease.

(e) Prior Treatment Methods, Including “Low Carbohydrate” Diets For Weight Loss

It should be noted that the primary origins or causes of the diseases mentioned above are unknown and, consequently, cures for them have yet to be discovered. Much of the research that is directed at them seems to be directed towards finding new drugs that can diminish their symptoms, while not eliminating their causes.

It is well known that many people, including Dr. Adkins, have previously recommended various forms of“low carbohydrate” diets. For example, in 1995 Sears (“The Zone,” Regan Books/Harper Collins, N.Y.) proposed a low carbohydrate diet that was less restrictive than Dr. Atkins' diet in that it did allow for some vegetable and fruit consumption.

Our research suggests that suppression of carbohydrates alone can only be part of an effective treatment for the above mentioned diseases; the regular intake of L-aspartic acid, as a diet supplement, is also essential.

(f) Prior Uses For L-Aspartic Acid

Several different uses of L-aspartic acid have been previously described in the field of medicine, but all of them are for different purposes than that proposed herein. These other uses include: (a) derivatives from L-aspartic acid that are proposed as sweetening agents, see U.S. Pat. Nos. 5,374,733, 5,430,182, and 4,612,195, (b) mixing L-aspartic acid with milk or dissolving it in aqueous solution to inhibit β-glucuronidase activity in breast feeding babies, in order to suppress serum bilirubin levels and the incidence of neonate jaundice, see U.S. Pat. No. 6,416,783, (c) several L-aspartic acid and L-glutamic acid chemical derivatives are known to be used as drugs for supposedly enhancing memory and learning, and also as antidepressants, see U.S. Pat. No. 5,723,494, (d) L-aspartic and other amino acid derivatives have been proposed as platelet aggregation inhibitors, see U.S. Pat. Nos. 5,053,392 and 5,399,570, (e) polypeptides containing L-aspartic acid and intermediates as anti-ulcer agents, see U.S. Pat. No. 4,012,367, and (f) as anti-viral agents, see U.S. Pat. No. 5,491,135.

Thus, despite the prior art, the opportunity still appears to exist for one to identify how L-aspartic acid can be used to treat a group of diseases (e.g., obesity, diabetes and hypertension) that share the characteristic that their victims' bodies exhibit abnormal amounts of fat storage.

3. Objects and Advantages

There has been summarized above, rather broadly, the prior art that is related to the present invention in order that the context of the present invention may be better understood and appreciated. In this regard, it is instructive to also consider the objects and advantages of the present invention.

It is an object of the present invention to provide a dietary supplement and its method of use that will decrease the number of people that exhibit the characteristic of having their bodies suffer from abnormal amounts of fat storage.

It is another object of the present invention to provide a dietary supplement for the treatment of a wide range of health problems that are associated with one's cells having a poor metabolic capability to process fat. Examples of such health problems include: obesity and the related health conditions that are aggravated by obesity, type-2 diabetes, hypertension, atherosclerosis, migraines and headaches, menstrual cramps, cholesterol excesses, multiple sclerosis and Alzheimer's disease, anemia, high blood uric acid levels, ketosis, tobacco and other drug addictions, and digestive problems.

It is yet another object of the present invention to show how the effectiveness of current low carbohydrate diets for weight loss may be greatly improved upon.

It is still another object of the present invention to call into question the validity of current paradigms for energy metabolism and nutrition, and to advocate their replacement by new paradigms which provide a better understanding of these matters.

Using these new paradigms of energy metabolism and nutrition, it is an object of the present invention to identify how a whole family of widely known health problems can be more effectively treated or prevented.

It is also an object of the present invention to show how an existing compound (e.g., L-aspartic acid) can be put to a new use that holds great promise for improving the treatment or prevention of a whole family of widely known health problems.

These and other objects and advantages of the present invention will become readily apparent as the invention is better understood by reference to the accompanying summary, drawings and the detailed description that follows.

### SUMMARY OF THE INVENTION

Recognizing the opportunity the identification of additional uses for L-aspartic acid, the present invention is generally directed to exploiting this opportunity and thereby contributing new treatment method for a wide range of health problems.

In a preferred embodiment, the present invention is a method or process of treating or preventing one's health problem that are associated with one's cells having a poor metabolic capability to process fat. Examples of such health problems include obesity and the related health conditions that are aggravated by obesity, type-2 l I diabetes, hypertension, atherosclerosis, migraines and headaches, menstrual cramps, cholesterol excesses, multiple sclerosis and Alzheimer's disease, anemia, high blood uric acid levels, ketosis, tobacco and other drug addictions, and digestive problems. In such an embodiment, this method includes the daily ingestion of a therapeutically effective dosage of an anaplerotic precursor and the avoidance in one's diet of starchy foods.

In a further embodiment, this anaplerotic precursor is L-aspartic acid, or a pharmaceutically acceptable analog thereof, that is ingested at the rate of 6-12 g/day while restricting from one's diet the intake of starchy foods.

In a still further embodiment, we introduce the concept that such health problems may be treated or prevented by one's adherence to what we call a “compensated diet.” We define such a diet to be one that contains sufficient amounts of anaplerotic precursors so that one's main pathway of cellular energy metabolism is not by glycolysis, but by fatty acid degradation (β-oxidation) coupled with the Krebs' cycle. One can achieve such a cellular energy metabolism by following a low starch diet and supplementing it, as necessary, with adequate amounts of anaplerotic precursors (e.g., glycogenic amino acids, especially L-aspartic acid).

Yet another preferred embodiment of the present invention is a compound for treating or preventing health problems that are associated with one's cells having a poor metabolic capability to process fat. Preferred forms of this compound are L-aspartic acid or a pharmaceutically acceptable analog thereof. Recommended daily dosages are 2-20 g depending on the metabolic capability on one's cells to process fat.

Thus, there has been summarized above, rather broadly, the present invention in order that the detailed description that follows may be better understood and appreciated. There are, of course, additional features of the invention that will be described hereinafter and which will form the subject matter of the claims to this invention.

### BRIEF DESCRIPTION OF THE DRAWINGS

FIG. 1 illustrates the main pathways of energy metabolism.

FIG. 2 illustrates the relationships among various diseases that are related to a cell's poor metabolic capability to process fat and its eventual accumulation in one's body.

FIG. 3 illustrates the main metabolic interactions among Krebs' cycle intermediates and anaplerotic pathways.

FIG. 4 illustrates the chemical non-enzymatic reaction of protein glycosylation: addition reaction driven by a nucleophylic attack of an amino group from a protein on the carbonyl group of glucose.

FIG. 5 shows the body mass and fat reductions for a 36 year old man who participated in the research studies for the present invention. His weight was 130 kg when starting with our treatment, and had 53.5 kg of fat (41.1% of the body mass). After 290 days of treatment, his weight was 83 kg (he reduced 47 kg) with 19.1 kg of fat (23% of the body mass). This plot demonstrates that the reduction of fat percentage was in a close correspondence with the body mass reduction, which suggests that the weight loss was not degenerative.

FIG. 6 illustrates the progress of glycemia during our treatment of type-2 diabetes in a 53 year-old man (weighted 95 kg and had 24% fat-percentage) who had been suffering from type-2 diabetes for over 10 years before beginning our treatment.

FIG. 7 illustrates the progress of glycemia (●) and insulin takings in the morning (●●) and in the evening (σσ) during our treatment of a 58 year-old man (82 kg, 31% fat) with type-2 diabetes for over 10 years.

Table 1 presents the results achieved by 13 patients who participated in the obesity portion of the clinical research that formed the basis for the present invention.

Table 2 presents the results achieved by 20 patients who participated in the type-2 diabetes portion of the clinical research that formed the basis for the present invention.

Table 3 presents the results achieved by 250 patients who participated in the hypertension portion of the clinical research that formed the basis for the present invention.

### DESCRIPTION OF THE PREFERRED EMBODIMENT

Before explaining at least one embodiment of the present invention in detail, it is to be understood that the invention is not limited in its application to the details of the methods set forth in the following description or illustrated in the drawings. The invention is capable of other embodiments and of being practiced and carried out in various ways. Also, it is to be understood that the phraseology and terminology employed herein are for the purpose of description and should not be regarded as limiting.

Disclosed below is what we believe to be a general solution to and treatment for the previously described health problems of obesity, type-2 diabetes, hypertension, etc. Additionally, we believe that the treatment methods disclosed herein will also be applicable to a large number of other health concerns and diseases, including: migraines and headaches, anemia, high blood uric acid levels, ketosis, tobacco addiction, multiple sclerosis, Alzheimer's disease and, even cancer.

We begin the present disclosure by providing a theoretical basis for our proposed treatment methods. To do this it proves useful to recall the current paradigms for energy metabolism and nutrition.

Current Paradigm for Energy Metabolism and Nutrition

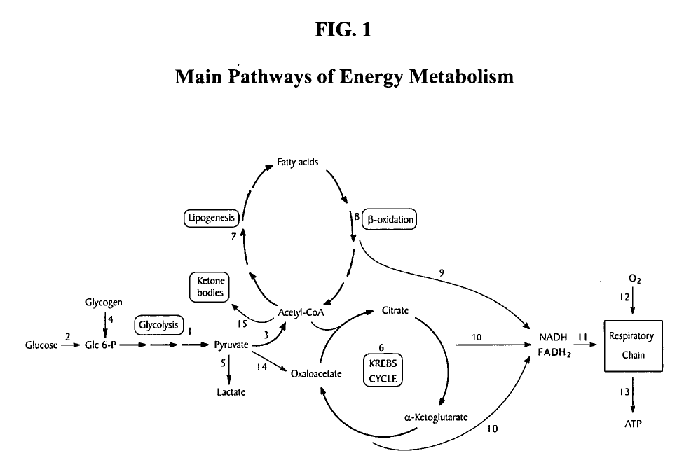

The system of metabolic reactions, pathways and physiological processes for producing energy is called “energy producing metabolism” (or “energy metabolism”) and are shown in FIG. 1. Oxidative energy metabolism comprises three phases: (a) the conversion of glucose or fatty acids into acetate, as acetyl-CoA, (b) Krebs' cycle, which accounts for the full oxidation of the acetate residues yielding reduction equivalents as NADH or FADH₂, and (c) respiratory chain and oxidative phosphorylation, where these reduction equivalents are oxidized by molecular oxygen producing water.

Energy released in these reactions is used to make ATP, the general central energy molecule of cellular metabolism. The first phase in this process can occur directly in all cases, but the subsequent oxidation of acetate in the Krebs cycle needs oxaloacetate as the feeder. Thus, the control of oxaloacetate concentration is a way to control the activity of this process.

Much of the accepted current understanding of this process is summarized in the following statements:

1. Glycolysis is the primary pathway of energy metabolism, as carbohydrates (glucose) are the primary energy fuel. Glycogen is the carbohydrate store material. Liver glycogen plays a role for blood glucostasis, while muscle glycogen plays a specific role as an energy source of each individual muscle fiber.

2. Fat metabolism is a secondary pathway that works only in certain tissues, and only under certain circumstances. It can be used in some cases as alternative energy pathway. Excess of fat is stored in the adipose tissue, and it is in principle available to be used for energy requirements of cellular metabolism.

3. Anaplerosis (synthesis of oxaloacetate to feed Krebs' cycle) is mainly fulfilled by pyruvate, which is produced from glucose metabolism.

4. Adrenalin is the stress hormone. Its role is to increase the blood glucose concentration releasing it from liver glycogen, in order to guarantee the glucose as energy fuel is available from the blood to be used for all tissues; it also activates muscle glycogen depletion allowing fast muscle activity.

5. Glucagon is also a hyperglycemic hormone, but its role is mainly as a component of homeostatic mechanisms, to increase the blood glucose level which could be low due to hypoglycemic conditions.

6. Insulin is a hormone that regulates the normal consumption of glucose by cells.

This paradigm of energy metabolism is the basis on which the central paradigm of macronutrition is built. This is summarized in the so-called food guide pyramid and can be summarized by the following guidelines or principles:

7. Carbohydrates must be the basis of food, as they are considered to be the basis for energy metabolism. The so called slow digestion carbohydrates (rich in starch) are much better than the so called fast digestion carbohydrates (rich in sugar).

8. Protein must be restricted as it is supposed that the need for them in metabolism is very low.

9. Fat must be avoided because it is considered that carbohydrates are better than lipids as fuels, and fat excess causes a wide number of metabolic diseases such as obesity, type-2 diabetes and cholesterol excess. Saturated fat must be especially avoided, while unsaturated fat should be consumed in moderate amounts.

10. Fiber (carbohydrates which cannot be digested, usually cellulose from vegetables) is necessary to help the digestion as it gives more surfaces for digestion improving the work of the enzymes in the digestive tract. Thus, a certain amount of fiber should be included in every meal.

11. Three meals per day are generally considered the minimum number that one should consume every day, but many nutritionists recommend five or six. It is believed that, in general, the number of meals consumed can be as important as the total number of calories.

12. The number of calories consumed per day should be controlled. It will be usually between 2,000 and 4,000, according to the amount of physical exercise that one gets. Thus, one's food should be chosen mainly according to its caloric content. Fat has a high calorie content (e.g., 9 Cal/g), while carbohydrates has a low calorie content (e.g., 4 Cal/g).

13. Physical exercise is highly recommended as a way to burn any excess of calories that are consumed.

Problems With These Paradigms of Energy Metabolism and Nutrition

Although these paradigms of energy metabolism and nutrition are widely accepted, our study of these matters suggests that they are incorrect. We note the following observations that are inconsistent with these paradigms:

1. Our earlier or wildfoods were not rich in carbohydrates—Foods rich in carbohydrates (starch) are artificial, as they have been strongly modified from their naturally occurring forms by the agricultural processes that man has developed. This includes the wheat that is used in bread, biscuits or pasta; other cereals, like corn and rice, and potatoes, beans, lentils and chickpeas whose content in carbohydrates is extremely high, around 60-80%. Meanwhile, the seeds from wild plants have smaller carbohydrate contents.

2. Human's poor ability to digest carbohydrates—The human stomach has a very poor capacity to digest carbohydrates, so it must be performed by hydrochloric acid (HCl), making a chemical (non-enzymatic) digestion. This means that HCl, a product whose role in digestion should be regulatory, is forced to work as a digestive catalyst thereby requiring large amounts of it which can itself create health problems.

Rodents (rats and mice) are perhaps the only wild animals strongly adapted to eat large amounts of carbohydrates. This is a very particular case in Nature, and its existence is explained by noting that rodents developed their energy metabolism to a greater extent by natural selection as they came to inhabit the new niches of wheat fields and barns that existed after modern agriculture was invented. While the human stomach has a very poor amylase activity, this activity is very high in rodents.

Although rodents are poor experimental models from which to study human nutrition and energy metabolism, they have been used for this purpose and such experimental work has helped form part of the basis for our current energy metabolism and nutrition paradigms.

3. High blood glucose concentrations & the need for continuous insulin secretion—A diet extremely rich in carbohydrates causes a high blood glucose concentration that stimulates high insulin secretion, which forces the use of glucose by several specific tissues. The continuous absorption of glucose during several hours after eating places the organism in an undesirable situation that is similar to that of a type-2 diabetic (i.e., with high blood glucose and insulin levels, and forcing a continuous insulin secretion).

4. Caloric intake control is ineffective for controlling one 's percentage of body fat—Despite trying to control our diets and food consumption according to the current nutrition paradigm, the populations of the world's industrialized nations continues to get fatter. The present paradigm contains no clear statement about the different ability of one's metabolism to use fat or carbohydrates as energy fuel. The body's different anaplerotic needs and a number of hormonal effects determine a clear qualitative distinction for the metabolic consumption of fat or carbohydrates.

5. Recommendations for number of meals per day do not consider details of one's metabolic activity—Several aspects of metabolic activity are submitted to circadian rhythms, so the destination and use of the food can be quite different according to the time it is ingested along the day. On the other hand, digestion stimulates secretion of gastrointestinal hormones that participate in metabolic regulation; furthermore insulin is secreted after eating—as the meal is usually rich in carbohydrates—enhancing a forced entry of glucose in some specific tissues with different metabolic ends.

6. Glycolysis shouldn't be the primary pathway for energy metabolism—Glycolysis is a fast energy pathway that allows the rapid production of ATP, yet most muscle contractions, such as contractions for standing or walking, are non-fast movements that usually must be maintained for longer durations. Fatty acid oxidation is a pathway more appropriate to support the regular movements of our body, while glycolysis is an emergency pathway that is more appropriate for fast muscle contraction movements.

7. Insulin as the hormone that controls the regular consumption of glucose by cells is implausible—There is really no data or any logical reason that supports the hypothesis that “insulin is the hormone that controls glucose consumption;” but, instead, there are many observation that strongly suggests that insulin plays a very different role (i.e., insulin is a hypoglycemic hormone that prevents glucose excesses by converting it into fatty acids which promotes obesity). For example:

(a) As a way to supply energy to cells, glycolysis activity cannot depend on a hormonal (external) signal, and less on a hormone whose lifetime is as short as a few minutes; the consumption of glucose by each cell to satisfy its energy needs must be regulated by internal signals, which will depend on the cell's particular energy necessities,

(b) There are many hyperglycemic hormones, including glucagon, adrenalin, (all cathecolamines), cortisone, cortisol, (all glucocorticoids), growth hormone, adrenocorticotrophic hormone (ACTH) and thyroxine, which increase the blood glucose level, whereas insulin is the only hypoglycemic hormone—the hormone driven removal of glucose from blood is not a process to supply energy fuel to cells, but more probably a means for cleaning the blood,

(c) While each hyperglycemic hormone is secreted in response to a different specific stimulus, which represents a specific metabolic necessity, regulatory pathways that produce pancreatic secretion of insulin depend solely on the blood glucose level—thus, the role of insulin, as it is secreted, is not related to the energy needs of the cells, but strictly with the blood glucose level—insulin is not a hormone for the cells, but for the blood, as its purpose is not related with cellular metabolism, but with blood cleaning,

(d) Up to 12 different glucose transporters (GLUT) have been identified (e.g., GLUT1 is the most ubiquitous, whereas GLUT3 is found only in the brain), but only GLUT4 (found in muscle, heart and adipose tissue) is insulin responsive—thus, most cells do not need insulin to consume glucose,

(e) Brain cells are assumed to not be capable of using any other fuel than glucose; however the brain's neurons do not need insulin as evidenced by the fact that the brain's glucose transporter (GLUT3) is not activated by it,

(f) Although muscle cells are among the most sensitive to insulin as they have the glucose transporter GLUT4, muscle glycolysis does not actually need it to work; it is well-known that physical exercise decreases one's blood glucose levels—people suffering from diabetes know that when they exercise their intakes of insulin or hypoglycemic drugs can be reduced,

(g) Insulin is also a metabolic regulator in that it activates fat biosynthesis in liver and adipose tissue (the two main tissues where fatty acid and triacylglycerol biosynthesis occur) by activating the gene expression for acetyl-CoA carboxylase, fatty acid synthase, and glycerol 3-phosphate acyltransferase; insulin also decreases the gene expression of pyruvate carboxylase, which is another effective way to enhance fatty acid biosynthesis,

(h) A diet highly rich in carbohydrates promotes fat synthesis and storage as carbohydrates are mostly converted into fatty acids; insulin is not a hormone that regulates cells' glucose consumption, but a hypoglycemic hormone that prevents glucose excesses by converting it into fatty acids which can promote obesity,

(i) The very short lifetime of insulin (a few minutes), suggests that its role is for emergency uses—insulin forces glucose's quick consumption in order to reduce the blood glucose level when other hormones (mainly adrenalin) have raised it too high,

(j) The origin of our prior misunderstanding of the role of insulin is probably due to the observation that the capacity of cells to consume glucose is poor, and that it could be increased with the influence of insulin—this led scientist to think that the activity of glycolysis should be much higher, and that insulin was necessary to maintain such higher rates—This mistake is surely one of the causes of the lack of understanding of type-2 diabetes, a disease for which science has yet to find a solution—the new paradigm we are presenting herein suggests new ways to fight this disease.

8. The liver's role in distributing glucose to the cells suggests that our diets should consist of fewer carbohydrates. Other than for muscular activities, carbohydrate metabolism is driven by the liver functioning to distribute glucose to the cells. The liver's capacity to store glycogen is a reference point on which the regular traffic of glucose can be calculated. The contribution of food carbohydrates to replenish liver glycogen is estimated at less than 50%, the rest being by the indirect way (gluconeogenesis) from amino acids. If the daily carbohydrate needs of a sedentary person are about 20% of the total capacity of liver glycogen (about 120 g for a person weighing 70 kg), such a person's daily carbohydrate needs are only 10-20 g, as opposed to the 500-1,000 g that are consumed by most people.

9. Hunger pangs are not due to an empty stomach but a response to the anxiety provoked by carbohydrate addiction. Many people apparently believe that hunger pangs are felt when the stomach is empty. However, wild animals that eat only once a day have empty stomachs for most of the day and apparently do not suffer hunger pangs—since humans' stomachs are similar to those of wild animals, why should ours be assumed to function differently? It is well known that carbohydrates—not only sugar—are a very addictive food that impels people to eat more of them. In effect, hunger or anxiety is more likely our body's demand for carbohydrates. When human beings invented agriculture and the high carbohydrate foods that it yielded, they, in addition to developing a way to fight the nutrition, started us on our addiction to carbohydrates.

10. The problem of obesity is not just one of the summation and subtraction of calories from one's diet. Apart from the excess of carbohydrates in our diets and their inevitable conversion into fat, the most important problem behind obesity is the inability of energy metabolism to consume fat. To determine the caloric value of a diet according to one's daily energetic needs would be right only if fat could be freely consumed. The fact that a person with a significant fat excess in the body still needs 1s a continuous intake of food for their daily energetic needs demonstrates that this person has a problem with consuming their excess fat for energy purposes. The first goal in fighting obesity is to find a way that will enable the human body to consume its excess fat.

11. Anaplerosis (the synthesis of oxaloacetate to feed Krebs' cycle) is dependent on factors other than just the availability of pyruvate from glucose metabolism. Consider that:

(a) Insulin inhibits pyruvate carboxylase activity by reducing its genetic expression. Its inhibition clearly means that under those circumstances the Krebs' cycle is poorly fed—acetyl-CoA coming from carbohydrates has to be converted into fatty acids. Not only does insulin promote fat biosynthesis, by a direct activation of lipogenesis, but it also prevents the oxidation of acetyl-CoA.

(b) As previously discussed, while carbohydrates can be consumed directly (without the support of any other carbon pathway), fat cannot be directly consumed since it needs a complementary anaplerotic pathway from another carbon source. It has been always assumed that glucose can play this role as the main oxaloacetate precursor via pyruvate carboxylase—thus, carbohydrates can promote fat degradation. However, observation of the obesity within populations whose diets' are based on massive amounts of carbohydrate consumption seems to call into question whether carbohydrates actually serve this purpose. The regulatory mechanism that explains the poor role carbohydrates can play as anaplerotic source is that insulin inhibits the expression of the pyruvate carboxylase gene. Thus, a diet with high carbohydrate content highly favors fat synthesis through two complementary ways: (i) it enhances fatty acid and triacylglycerol biosynthesis, and (ii) by blocking the anaplerotic role of glucose, pyruvate must be converted into acetyl-CoA, driving the glycolytic flux toward fatty acid biosynthesis.

(c) Physical exercise is known to be a way to promote direct fat consumption and to enhance carbohydrate degradation thereby increasing their use as a fuel for energy metabolism. Our research leads us to believe that physical exercise also can neutralize the antianaplerotic effect of insulin on pyruvate carboxylase, and/or promote other anaplerotic ways from pyruvate (e.g., through the malic enzyme).

The observations have led us to conclude:

The food which most human beings consume has a huge excess of carbohydrates, whose results include: (a) hindered digestion, (a) a forced metabolism in the liver and adipose tissue that convert carbohydrates mostly into fat, and (c) persistently high level of blood glucose, which provokes a continuously high secretion of insulin to clean excess glucose from the blood.

Our main metabolic problem is our body's inability to consume fat.

The main problem of type-2 diabetes is not the cell's insulin resistance, but their inability to burn glucose.

The strategy to fight diabetes should be driven towards recovering the body's ability to burn glucose, and not towards recovering the body's sensitivity to insulin.

Our current paradigm of energy metabolism is clearly inadequate and needs to be replaced.

Our current paradigm of nutrition and its food guide pyramid, with its emphasis on carbohydrate consumption, is clearly wrong and needs to be replaced.

Proposals for New Paradigms of Energy Metabolism and Nutrition—Theoretical Basis for the Present Invention

These observations have led us to propose a new paradigm of energy metabolism and nutrition. The new paradigm we propose is summarized in the following statements:

1. Fat degradation. The main pathway of cellular energy metabolism should not be glycolysis, but fatty acid degradation (β-oxidation) coupled with the Krebs' cycle.

2. Glycolysis. Glycolysis should be an emergency pathway of cellular energy 1I metabolism for those special processes that require a fast source of energy, and its use must be especially activated by specific regulatory signals.

3. Carbohydrates. Diets with large amounts of carbohydrates (e.g., cultivated tubers (potatoes, yams), cultivated forms of tall cereal grasses (wheat, oats, corn, rice) and the products made from them (flour, breads, biscuits, rolls, pasta, pizza, cakes, pies, buns, sweet rolls), cultivated pulses (peas, beans)) must be avoided because they cause many problems in fat metabolism and do not yield the energy expected benefits.

4. Proteins. Solid-food-containing meals should contain should include some food rich in proteins (e.g., meat, chicken, ham, fish, seafood, eggs), and one's main meal of the day should contain a significant amount of proteins (e.g., 30-40 g of protein for one weighing 70 kg). Solid-food-containing meals should be a source of amino acids, otherwise one's metabolism is forced to take them from muscle degradation.

5. Anaplerosis. Full metabolic oxidation of fatty acids needs an appropriate intake of anaplerotic precursors (source of oxaloacetate) to guarantee the incorporation of acetyl-CoA in the Krebs cycle (the main natural pathway for this is by the coupling of glycolysis with pyruvate carboxylase, but this pathway cannot work with a starch- or carbohydrate-rich diet; an alternative anaplerotic pathway is provided by various glycogenic amino acids—our results show that L-aspartic acid is the most effective).

6. Compensated diet. We advocate a “compensated diet” which we define as one that contains sufficient amounts of anaplerotic precursors (compounds capable of yielding oxaloacetate) to account for a diet's content of fat or substances that are going to be converted into fat. Carbohydrates uncompensated one's diet (i.e., a diet rich in carbohydrates promotes large insulin secretion that enhances fatty acid synthesis and blocks anaplerosis. Glycogenic amino acids, especially L-aspartic acid, are good anaplerotic precursors that can be used to compensate one's diet. Proteins are a natural source of such acids and also serve to help compensate one's diet by balancing its preferably low content of fat and carbohydrates (i.e., foods rich in starches should be avoided; vegetables, fruit and milk can be included in our recommended diet when they are combined with protein-rich foods and additional compensation, if necessary, is provided by recommended amount of anaplerotic precursors).

7. Fiber. A key reason why it is suitable to include fiber in the diet is to make the digestion of carbohydrates easier. For a low carbohydrate diet, fiber consumption can be proportionately reduced. However, weight loss programs following our treatment methods should include sufficient amount of fiber to aid one digestion.

8. Vitamin C (ascorbic acid). Results of our clinical trials suggest that it is important to guarantee a daily intake (e.g., 0.5-1.0 g) of vitamin C. This is a key reason (along with its fiber content) why uncooked fruit is recommended in our diets.

9. Insulin. Insulin is not a hormone that regulates the normal use of glucose by cells, but rather an emergency hormone, whose role is to clean the blood of an excess of glucose (which can be caused by the consumption of carbohydrates). A diet rich in carbohydrates provokes large releases of insulin, which blocks fatty acid consumption promoting its synthesis.

Application of the Present Invention to Develop Improved Treatments for Assorted Health Conditions

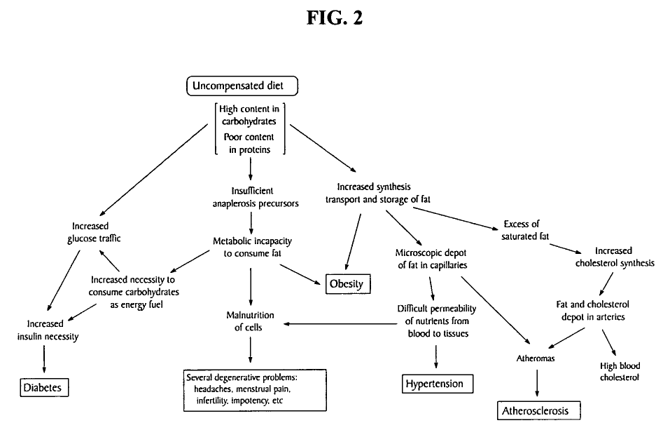

Our new proposed paradigm of energy metabolism and nutrition suggests improved treatment methods for an assortment of health conditions, see FIG. 2, including:

1. Obesity

Rather than a hypercaloric diet, malfunctioning of our hunger regulatory mechanism or a lack of physical exercise, we believe that the primary cause of obesity is a diet extremely rich in carbohydrates (their consumption yields high blood glucose levels which are subsequently reduced by insulin releases which serve to remove glucose from one's blood by converting it into fatty acids that block the use of glucose as an anaplerotic precursor). Carbohydrates are very addictive—the 1I repeatedly high blood glucose level yielded by carbohydrate consumption can promote an addiction to them.

The excess of carbohydrate consumption in one's body induces persistent excesses of insulin secretion and progressively reduces pyruvate carboxylase activity, 15 causing a progressive loss of the anaplerotic capability in cellular metabolism. Thus, as people put on weight, their pyruvate carboxylase activity is more and more reduced, and with it their anaplerotic capability. Thus, a sudden elimination or strong reduction of carbohydrates in one's diet is not enough to solve one's weight problem, as the person continues to have a lack of anaplerosis.

Physical exercise can reduce this antianaplerotic effect (the failure of pyruvate carboxylase gene expression); however, since that effect occurs at the gene expression level, it needs some time to have the desired effect. So therapy to fight obesity based on physical exercise requires that the exercise be continued over a long duration, and, even then, it may be not enough to achieve a significant reduction of fat.

A “compensated diet” (i.e., one that contains sufficient amounts of anaplerotic precursors so that one's main pathway of cellular energy metabolism is not by glycolysis, but by fatty acid degradation (β-oxidation) coupled with the Krebs' cycle [achieved by following a low starch diet and supplementing it, as necessary, with adequate amounts of anaplerotic precursors (e.g., glycogenic amino acids, especially L-aspartic acid)]) is necessary to avoid fat synthesis and storage. The problem of obesity is not an excess of food, but the inability of one's body to use that food properly in cellular metabolism.

Treatment—Since obesity is associated with a lack of anaplerotic resources, the immediate and more important problem is the recovery of this function. A drastic reduction, or better a full elimination, of foods rich in starches from the diet is a necessary condition for achieving this recovery (as the first goal is to avoid the high insulin secretions that are driving metabolism toward fat synthesis). However, as the recovery of pyruvate carboxylase activity can be a long, slow process, it is necessary to provide in one's diet a supplementary anaplerotic precursor in order to expedite a weight loss program.

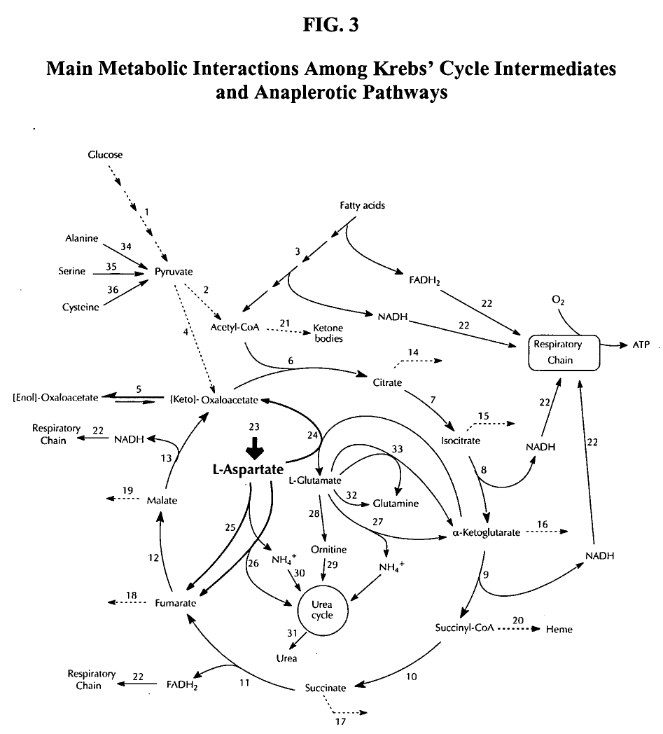

Considering all the possible metabolic interactions that can provide for the desired anaplerotic function, we have concluded that L-aspartic acid appears to be the most effective product to play this role, as it is the most closely related with 1 5 oxaloacetate, and can enhance the anaplerotic function of the Krebs' cycle in several different ways. See FIG. 3. This consumption of L-aspartic acid can probably be decreased with time as pyruvate carboxylase recuperates its activity so as to allow one's metabolism to recover its natural anaplerotic function. Then the treatment by L-aspartic acid intake could be discontinued, provided that one's diet is well compensated.

2. Diabetes

Diabetes is a disease characterized by an abnormal metabolism, storage and distribution of glucose, which is manifested by high blood glucose levels. This makes a regular administration of drugs to reduce it necessary. There are two different kinds of diabetes (i.e., types -1 and -2), according to the state of P-cells of pancreatic islets that are the cells that produce insulin. Type-1 diabetes is a disease caused when the immune system attacks β-cells of pancreatic islets with the result that those afflicted lose their source of insulin, which has to be administrated on a regular basis, two or three times per day. Meanwhile, patients (usually those over 45) with type-2 diabetes have normal levels of insulin, but this hormone does not produce its expected effect because a number of cells of the body are insensitive to it. Type-2 patients have to reduce their blood sugar levels with different kinds of drugs, and even with injections of insulin.

In our discussion of type-2 diabetes, we talk about diabetic cells and normal cells, instead of diabetes in a general sense, because in patients suffering from this disease, not all their cells have the same degree of damage. We refer to diabetic cells as those that have lost their sensitivity to insulin or have it very diminished.

How does a normal cell becomes diabetic or insensitive to insulin? We propose that insensitivity to insulin is expected response of the cell to an external command (executed by insulin) to force it to intake an excess of glucose in order to clean the blood. This proposal immediately suggests that a person suffering from type-2 diabetes, being under drug or insulin treatment, is permanently putting their normal cells at risk of becoming diabetic too, permanently increasing the amount of diabetic cells in their body.

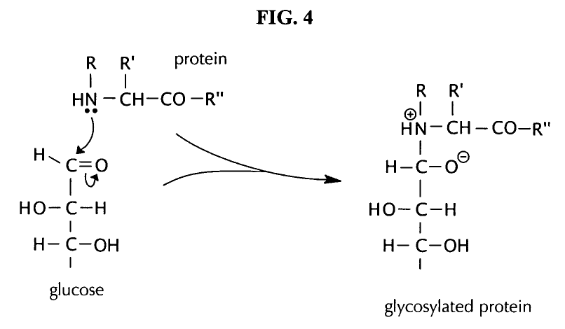

As aldehyde, glucose is a strong chemical reagent that attacks amino groups of proteins (see FIG. 4). These reactions spoil proteins, and are, obviously, non-enzymatic. Glycosylated hemoglobin is a statistic index of damaged proteins in the whole body. The insulin resistance typical of type-2 diabetes can be, thus, understood as a logical defense of the cells against the massive aggression of glucose forced by the high insulin levels. Thus, insulin resistance is not a bad property for the cell, but a mechanism to avoid an excess of glucose, which becomes toxic.

Treatment—An increase in fatty acid metabolism will reduce the importance of glycolysis, and hence one's need for insulin. This can be achieved by one's adherence to a “compensated diet.” Results shown below demonstrate that this treatment method works. Although type-1 diabetes is quite different from type-2, we have found that this treatment also yields good results for type-1 patients. Reducing the body's need for insulin is effective for both kinds of diabetes.

3. Hypertension

We believe that our observations, conclusions and ideas, as presented above, also can contribute to one's understanding of hypertension. It can be noted that there is no consensus on the primary cause of hypertension and the mechanism that can cause this disorder, but general agreement seems to exist that one's excess of body fat is a very important risk factor for hypertension.

We believe that the increased accumulation and transport of fat by blood vessels provokes microscopic deposits of fat on the inner walls (atheromas) of the capillary endothelia, reducing the permeability of the capillary walls, for the nutrients that should go to the tissues. This can provoke two main problems: (a) an increase in blood pressure, and (b) malnutrition of the tissues that should be irrigated by these blood vessels, since the nutrients supplied by the blood cannot reach their destination. Hypertension is, thus, not a disease, but a symptom of other primary problems.

It is important to state that an excess of fat in the body does not necessarily mean an apparent excess of weight. We observed many patients who had a very high excess of fat (more than 30% in men and more than 40% in women) without appearing to be obese or having body weights that suggested obesity.

Two major kinds of drugs are used to fight hypertension are: (a) inducers of vessel dilatation, that expand the capillary walls, allowing the passage of nutrients to the tissues, and (b) β-blockers, which make the heart pump more slowly. The problem of the first group is that they weaken the capillary walls, and can promote their break down. The second group can reduce hypertension, but do not solve cell malnutrition, so the problem is aggravated, provoking degenerative processes in the tissues that suffer from an insufficient supply of nutrients.

According to our theory, hypertension should be considered as a consequence of anomalous deposits of fat (outside the adipose tissue) on the walls of capillaries, probably due to problems in fat transport in the blood or to an excess of fat transport in the body.

Treatment—An increase in fatty acid metabolism should reduce the problems of excess fat transport in the blood. We believe that this can be achieved by adhering to a “compensated diet.”

4. Atherosclerosis

An atheroma is a deposit of lipid containing plaques on the walls of an artery. Atherosclerosis is a form of arteriosclerosis characterized by the deposit of atheromatous plaque on artery walls. Such plaque has the effect of narrowing the lumen (channel) of the artery, thus restricting blood flow. This predisposes to a number of conditions, including thrombosis, angina, and stroke.

Treatment—Our prior disclosures suggest that an increase in fatty acid metabolism could reduce such fat deposit problems. This can be achieved by adhering to a “compensated diet.”

5. Migraines and Headaches

Our clinical trails and studies suggest two possible causes for migraines and headaches, both of which we believe can be closely related to microscopic fat deposits on the capillary walls: (a) local hypertension provoked by microscopic deposit of fat in the capillaries of the area; and (b) malnutrition of cells due to their incapacity to burn fat, with a consequent lack of energy.

The real cause of migraines and headaches might be a combination of these two factors, as one does not invalidate the other, and both of them are determined by the same primary cause: a high increase of fat synthesis with its consequent transport through vascular system that can provoke its uncontrolled deposit. We speculate that other health conditions (e.g., menstrual pains) may be caused by similar cellular incapacities.

Treatment—An increase in fatty acid metabolism that can be achieved by adhering to a “compensated diet.”

6. Cholesterol Excess

Excess of blood cholesterol is a very important high risk factor in a number of coronary diseases. An excess of cholesterol can result from: (a) a strong demand for its synthesis in the body; (b) the failure of cybernetic mechanisms that control its synthesis; and (c) a poor elimination of steroids as bile salts.

The main cellular role of cholesterol is to compensate for a poor ratio of unsaturated/saturated fat in the lipid bilayer of cell membranes which would otherwise result in the membrane being rigid. The lipid bilayer of animal membranes needs to have a soft fluid structure to allow it to function correctly as a fluid mosaic. This physical condition can only be achieved if it has enough unsaturated fat.

If the ratio of unsaturated/saturated fat available in the body to build membranes is low, the liver must compensate by synthesizing cholesterol. The main reason for cholesterol excess is, thus, not a high intake of it in the diet, but a high synthesis of saturated fat by our own organism, promoted by the excess of carbohydrates in the diet.

Our results demonstrate that a severe reduction or total elimination of carbohydrates in the diet largely reduces cholesterol levels in the blood, even increasing HDL (good) cholesterol and decreasing LDL (bad) cholesterol.

Treatment—Adherence to a “compensated diet.”

7. Multiple Sclerosis and Alzheimer's Disease

As discussed above, glucose is a highly toxic molecule because of its high reactivity power due to the carbonyl group of the aldehyde function in C-1. This makes it a good target for nucleophylic attacks from amino groups forming stable Schiff bases (see FIG. 4).

Thus, a high concentration of glucose in the blood (higher than 7 mM, equivalent to 125 mg/100 mL) can greatly increase nucleophylic attacks on amino groups of proteins including myelin proteins (it can be noted that the real meaning of glycosylated hemoglobin percentage, which can be tested in blood, is a statistical estimate of the whole percentage of damaged proteins caused by glucose toxicity). Different damaged proteins are thought to produce a number of diseases and health problems.

Alzheimer's disease (a mental illness characterized by the glycosylation of the myeloid precursor protein, a non-enzymatic reaction that seems to initiate the pathological process) and multiple sclerosis (a neurodegenerative disease characterized by a loss of myelin in the central nervous system, and caused by an apparently anomalous activity of the immune system; evidence is accumulating that glucose attacks some myelin proteins—it is recognized that people suffering from diabetes, which regularly produces a very high glucose concentration in the blood, have a high risk of suffering from multiple sclerosis) are speculated to be two extreme cases where the toxicity of glucose can be an important factor in initiating the pathological processes for these diseases.

Treatment—We propose that the first steps to fight these diseases should be to foster the recovery one's normal capacity to degrade fat (i.e., by adhering to a “compensated diet”). In addition, this treatment should be complemented with a regular intake of glycine, as we have previously shown in U.S. patent application Ser. No. 11/199,327, in order to reinforce the mechanical structures of the body.

8. Anemia

Anemia is the general name for a broad group of diseases characterized by the occurrence of some problem in the function of red blood cells to transport oxygen. There can be many causes for anemia, as there are many steps involved in the production of red blood cells.

In U.S. patent application Ser. No. 11/199,327, we have described the necessity of glycine as a very important nutrient to fight anemia. Glycine deficiency is probably the main cause in most cases of anemia, but in the light of the theory presented here, we speculate that malnutrition due to excess of carbohydrates and the incapability to use fat could be an important secondary cause of anemia.

Treatment—In addition to glycine as a dietary supplement, adherence to a “compensated diet.”

9. High Blood Uric Acid Levels

An excess of uric acid has heretofore been considered as a consequence of a large amount of red meat and seafood in one's diet. However, we believe that we have demonstrated that a high level of uric acid in the blood is a consequence of a diet based on carbohydrates.

When a diet is based on starch, energy metabolism cannot work correctly, and Krebs' cycle needs glycogenic amino acids to be fed. Blood albumin is a protein that stores glycogenic amino acids. Yet, when energy producing metabolism needs more glycogenic amino acids, they will come from one's skeletal muscles. As muscle cells are multi-nucleated cells, a large amount of nucleic acids will appear as a residue, which is then converted into uric acid.

Treatment—Adherence to a “compensated diet.”

10. Ketosis (A Pathological Increase in Ketone Bodies)

Under conditions of fatty acid degradation, if Krebs' cycle is not fed enough, it cannot incorporate all acetyl-CoA production properly. This excess of acetyl-CoA is deviated to the pathway of ketone bodies synthesis, and these compounds (acetoacetate, β-hydroxybutirate and acetone) are unloaded into the blood to be used by peripheral tissues. With the improved fatty acid degradation that our treatment methods yield, we would expect to see our patients show improvements in this condition.

Treatment—Adherence to a “compensated diet.”

11. Tobacco Addiction

Tobacco addiction is related to anxiety. The difference between obese people and smokers is that the first group is anxious to eat continuously, and the second group feels an anxiety to smoke. As we have demonstrated in our research, there is a close connection between both kinds of anxiety.

Treatment—Since L-aspartic acid dietary supplements control anxiety to eat in obese people, we speculated that they could also control one's anxiety to smoke.

12. “Pregnancy Diabetes” and Fetus Development

Pregnancy is a physiological state in which serious problems of glucose consumption and availability between the mother and the fetus can appear. A consequence of this conflict is the so-called “pregnancy diabetes” that some mothers suffer from in the last months of pregnancy. It is well-known that during pregnancy there are difficulties in feeding the Krebs cycle in both the mother and the fetus.

Treatment—Dietary supplements of L-aspartic acid can be the key to making the Krebs cycle work correctly in both mother and fetus, thereby reducing the risk of “pregnancy diabetes,” compulsive eating and ketosis.

13. Cancer

Two separate problems can be identified with cancer: its emergence and its 1I propagation. Many different factors can influence the transformation of a normal cell into a cancerous one, but all of them provoke some kind of cellular stress, which promotes anomalous mitoses, or a chain of mutations.

In U.S. patent application Ser. No. 11/199,327, we stated that glycine consumption can efficiently decrease the spread of cancer cells by reinforcing the connective tissue, and so hindering the invasive progress of cancer cells.

In the light of the postulates disclosed herein, it follows that malnutrition (which means high blood glucose levels and fat accumulation) can be a frequent cause of cellular stress. We suspect that the more frequent occurrences of cellular stresses can lead to greater incidences of cancer.

Treatment—As noted above, malnutrition can be treated by a diet that is low in carbohydrates and supplemented by the regular intake of glycogenic amino acids (e.g., L-aspartic acid). It follows that L-aspartic acid, as a product that efficiently fights cellular malnutrition, should therefore be considered as a potential product to prevent cell transformation into cancer.

14. Sexual Potency and Fertility

Obese people are known to often complain of problems related to sexual potency and fertility. Thus, we would expect that our weight loss treatment methods should also contribute to helping to remedy such situations.

Treatment—Weight loss programs based on adherence to a “compensated diet.”

15. Digestion Problems

Our bodies are known difficulties to have in digesting carbohydrates. Thus, we would expect that one following our recommended “compensated diet” would have fewer digestive problems.

Treatment—Adherence to a “compensated diet.”

Recommended Treatment Dosage and Protocols

The basis of the treatment for the diseases considered herein is a daily intake of L-aspartic acid or a pharmaceutically acceptable analog thereof, but the whole protocol includes the control of one's diet and eating habits. These matters are explained below.

1. Intake of L-Aspartic Acid

For the treatment of obesity, the regular recommended dosage of L-aspartic acid used in the clinical trials discussed herein was usually between 6 and 12 g per day, administrated in at least four doses of 0.5 to 3 grams (in powdered form, one small flat teaspoonful) at the following times: (a) early in the morning at breakfast time (around 8 a.m.), (b) mid-morning at 11 a.m. approximately, (c) early evening at about 5 p.m., and (d) at 8 p.m. or later at night. This is the basic schedule, but it can be rearranged according to the times of each person's meals. The aim is to have the Krebs cycle fed continuously, in order to make fat burning possible throughout the day.

However, we found it advisable to increase this intake in the case of extreme obesity. In these cases, the treatment has to be drastic to obtain a fast weight reduction (2-4 or even more kilograms or 4-9 pounds per week).

This required a program in which the patient would not have more than one full meal (i.e., with solid food) per week (see frequency of meals below). These patients must have a fifth L-aspartic acid dose in the early afternoon (even up to 20 g/day of L-aspartic acid), and can eat 2-4 servings of yoghourt (or yogurt) and also 200-400 ml fruit juice per day. These cases were under clinical control, which included blood analysis of uric acid, ketone bodies, glucose, cholesterol and other lipids every 2-4 weeks. Under this extreme program, if patients feel some kind of anxiety or hunger during the day, supplementary doses of L-aspartic acid must be taken as necessary to eliminate such sensations.

L-aspartic can be taken with fruit juice, which has sugar, to help to avoid hypoglycemia. The most important thing about this treatment is that under no circumstances should the patient feel hungry. If this is the case (even while taking extra doses of L-aspartic acid) patients should eat proteins (eggs, fish or meat) accompanied by some carbohydrates with no starch (such as vegetables, fruits and dairy products).

In some cases, the absolute elimination of carbohydrates can provoke an odd feeling during the first or second week (abstinence syndrome, see below). This feeling can be countered by ingesting food with a small amount of sugar, such as a piece of fruit. Yet, the best way to prevent it is to take L-aspartic acid with fruit juice. In this way, some sugar will be introduced into the patient's body, helping it to adapt to the new lower glucose levels in the blood.

For the treatment of tobacco addiction, we found it advisable to increase the frequency of the intake of L-aspartic acid to 10-12 small doses per day in order to control the anxiety to smoke. This does not mean taking more than 12 grams per day, but rather taking it in smaller doses (for example 0.5-1 gram hourly or every hour and a half).

Sometimes under special conditions of stress and inactivity, a feeling of anxiety can appear that can cause a person to eat compulsively, even at unusual times. In such cases of anxiety, L-aspartic acid should be taken more than 4 times a day, but always in small doses (for example 0.5-1 gram hourly or every hour and a half).

2. Amount and Frequency of Food Intake in Treatment of Obesity

The protocol used in our clinical trails included:

Breakfast—The first meal of the day should only be some liquid, for example, a small glass (100 mL) of fruit juice or a yoghourt with the regular dose of L-aspartic acid (0.5-3 grams), and an optional cup of white coffee.

Lunch—This should be the only full meal (with solid food) of the day, but it must totally avoid carbohydrates. The fact that there are no other full meals should not cause a problem once L-aspartic acid makes it possible for one to be capable of feeding on one's own fat.

Dinner—No solid food must be ingested in the afternoon and evening. This is especially important to achieve maximum fat expenditure, as the mechanism of fat burning works mainly overnight (see details below). In our trials, some of our patients decided to have their solid meal in the evening, whilst spending time with their family. It should be observed that the method we propose also works this way, but not as well as when nothing is eaten in the evening.

3. Special Protocol to Achieve a Faster Weight Loss

To make fat consumption faster we found that it was advisable to totally avoid solid food at night, before sleeping. Our best results were achieved if no solid food was taken after lunch.

The reason for this effect is probably a mechanism to save the stored fuel when other food is ingested. It probably works through hormonal secretion when solid food is retained in the stomach. We have seen that a little food ingested before sleeping prevents the desired high level of expenditure of fat at night.

4. Composition of the Full Meal

Our basic rules for the composition of the full meal are:

No carbohydrates.—Under the treatments disclosed herein, all food which is rich in carbohydrates (e.g., potatoes (fried potatoes, crisps, chips, boiled potatoes, mashed potatoes, etc.), all derivatives of wheat and flour (bread, pasta, pizza, etc.), any other kinds of cereals, including corn and rice, all kinds of pulses (plants that provide edible dried seeds: beans, chickpeas, lentils, kidney beans etc), all cooked carbohydrates containing bread or flour (croquettes, rissoles, etc.), all kinds of cakes (pies, tarts, buns, sweet rolls, bread rolls, biscuits, etc.) must be avoided and better eliminated completely from one's diet.

Protein—Foods rich in proteins (e.g., meat, chicken, ham, fish, all seafood) must be eaten in the day's one full meal. The reason for this rule is that eating and digestion stimulates metabolism, increasing amino acid traffic, so it is important for the meal to be a source of amino acids, otherwise these amino acids would come from muscle degradation. It should be noted that the composition of our recommended full meal is not what is often referred to as a strictly “high protein diet.” It is a meal that contains no starches; as vegetables, fruit and milk are included in our recommended diet, but these are always combined with foods rich in proteins.

Fiber—Because of the significant reduction in the quantity of solid food consumed under the diets recommended herein, it is good to eat some fiber (e.g., vegetables with low carbohydrate content) in order to promote the proper working on one's digestive tract. Fruit is also rich in fiber, but its consumption must be monitored carefully in certain cases, such as when patients are suffering from diabetes, since fruits have a large quantity of sugar.

Unsaturated fat—Fat is a normal and natural complement of our recommended diet. We recommend an amount of about 10-15 g per week of food rich in unsaturated fat.

Vitamin C.—The massive fat burning that occurs during the treatment of morbid obesity, as explained above, promotes obviously a high oxygen traffic through the respiratory chain, which will increase the probability that free radicals are produced. Thus, it is recommended that such patients take an extra dose of vitamin C, about 0.5-1 grams every day divided into two doses, as antioxidant, in order to keep the oxidative metabolism clean.

Micro Nourishment—Vitamins and minerals must be also consumed regularly. For very obese patients, following the diets recommended herein, we advise a regular intake of vitamins. We recommend a regular intake of B-complex of about 15-50 mg per day. Vitamin C has been mentioned above.

5. Protocol for Treatment of Diabetes

There is, in principle, no special protocol for patients suffering from diabetes (both types), which is different from the general one described above. Type-2 diabetes is usually associated with overweight and/or excess of fat in the body, so its protocol should not differ substantially from the general one presented above. With regard to type-I diabetes, since this treatment allows cells to consume less glucose, as it enhances their capacity to consume fat, it can reduce one's insulin dependence.

6. Further Considerations

Metabolic fat degradation can be simplified by means of a global chemical reaction with the following stoichiometry:
2[CH₂]+3O₂→2H₂O+2CO₂ (1)
where the structure of fatty acids is simplified as [CH₂]. This reaction represents the total oxidation of fatty acids by molecular oxygen to carbon dioxide plus water. Since there is a fixed stoichiometric relationship between [CH₂] and H₂O, the spent fat can be calculated from the amount of water produced, and it is not necessary to measure the two gases, O₂ and CO₂, as for stoichiometric effects, the global reaction can be considered a net conversion of fat into water with the fixed stoichiometric relationship of [CH₂]/H₂O=1.

Since the molecular weight of [CH₂] is 14, while H₂O is 18. The ratio 14/18 is, thus, approximately, the ratio of mass conversion of fat into water. This means that one liter of water (usually eliminated in urine) that does not correspond to water drunk means approximately 778 grams of fat burnt.

It is usually believed that a person could survive for several days without eating, but not without drinking water. However, our experimental results and the above reasoning suggests that people under this treatment can live for many days without drinking water or any other liquid, as the fat degradation produces enough water to fulfill the necessities of the body.

Moreover, we have seen that people under this treatment generate a large amount of urine without drinking the corresponding amount of water. Patients with obesity following this treatment were observed to have to get up one or two times during the night to urinate, which is a good indication that the desired fat burning mechanism is working. This amount of water unloaded gives an estimation of the amount of fat spent, according to the ratio 14/18 explained above. The results obtained demonstrate that large amounts of fat, about 200-600 g, can be burnt per day.

This much water production in our patients had another consequence. Since, under our recommended treatment, an obese body is effectively converted into a water-producing machine, it was observed that frequently no thirst was felt by the patients. Therefore, unconsciously, they drank less water or other liquids, and, with much less water circulating through their digestive tracts, they exhibited, in some cases, hard stools and difficulty with bowel movements. If this happens, more water should be drunk with meals to avoid constipation, even though no thirst is felt.

Under our recommended program, fat is literally burnt as this process is not dependent on physical exercise. This will obviously produce a great deal of heat, as no energy is spent on doing physical exercise. Thus, patients under this program will feel that their body is producing heat sometimes during the day (especially when suffering from obesity). Our results show that this heat generation occurs mainly at night, while the patient is sleeping. Heat is mainly felt in the legs and chest, in accordance with the location of brown adipose tissue, which often causes patients to remove blankets from their bed. This is another indication that the mechanism works, as fat burning occurs mainly at night while asleep.

The fact that a person under this treatment becomes virtually a water-producing machine, clearly suggests that L-aspartic acid is ideal for a number of situations where scarcity of water is a highly probable risk, and can lead to dangerous conditions. Examples of these are castaways, prolonged exploration campaigns or military missions, where water can be scarce as it is bothersome to transport.

Elimination of foods rich in carbohydrates can produce some small inconveniences manifested by episodes of weakness or feeling sick. This is because when eating food which is rich in carbohydrates our body is adapted to having high levels of glucose during the day, and when these level descent, a syndrome of abstinence can appear.

This abstinence syndrome can be fought by eating a sweet, fruit or just by taking additional small doses of L-aspartic acid with a sugary drink, such as fruit juice. In our trials, patients exhibited this syndrome only during their first two weeks of participation. When something sweet is eaten, it disappears almost immediately. In extreme cases, it may be suitable to prolong the adaptation period to three weeks by including a small amount of carbohydrates, instead of removing them suddenly from the diet on the first day of the treatment.

In our clinical trials, some of our patients were addicted to some soft drugs like coffee (caffeine), tobacco (nicotine), Prozac (fluoxetin), Trankimazin (alprazolam), Tranxilium (di-potasium chloride-acetate). In general, they were able to reduce their consumption of these materials while undergoing our recommended treatments. These results suggest that L-aspartic acid could also be a tool to help in eliminating stronger drug addictions. In tobacco release addiction programs, some small supplementary doses (0.5-1.0 g. every 30-45 minutes) may be necessary during the day to stop the patient's anxiety.

Results of Clinical Trials of Present Invention's Treatment Methods

Patients were examined once a week during the first three or four weeks, and thereafter, every ten or fifteen days. In every visit patients were weighed, and their fat was tested with a body fat monitor (model BF300, Omron Healthcare, Inc., Vernon Hills, Ill.). Blood analyses were carried out on all patients before starting our treatment and were repeated after three months.

Diabetic patients were told to take their values of blood glucose regularly (three or four times a day during the first months) until they achieved good blood glucose values. During the treatment, when it was seen that blood glucose levels were decreasing, the patients were instructed to reduce the doses of drugs or insulin, until removing them completely, to avoid hypoglycemia.

1. Obesity

Those suffering obesity or different degrees of being overweight were the most numerous group in this program, they being more than 1,500 whose ages varied from 18 to 83 years. Our clinical program started in May of 2002, thus, some of our patients have been people in our program for almost three years.

Table 1 shows a summary of some representative cases we treated for obesity. General observation that we have made regarding these trials include:

(a) Recommended treatments were seen to be effective for a broad range of patients, including both men and women having a wide range of weight problems (e.g., need to lose from 10 to more than 45 kg), and whose ages varied from 18-83 years.

(b) Patients generally reported no significant anxiety, hunger pangs or energy loss while on the recommended treatment programs, without the patient consuming a sedative or stimulant drug (e.g., amphetamines).

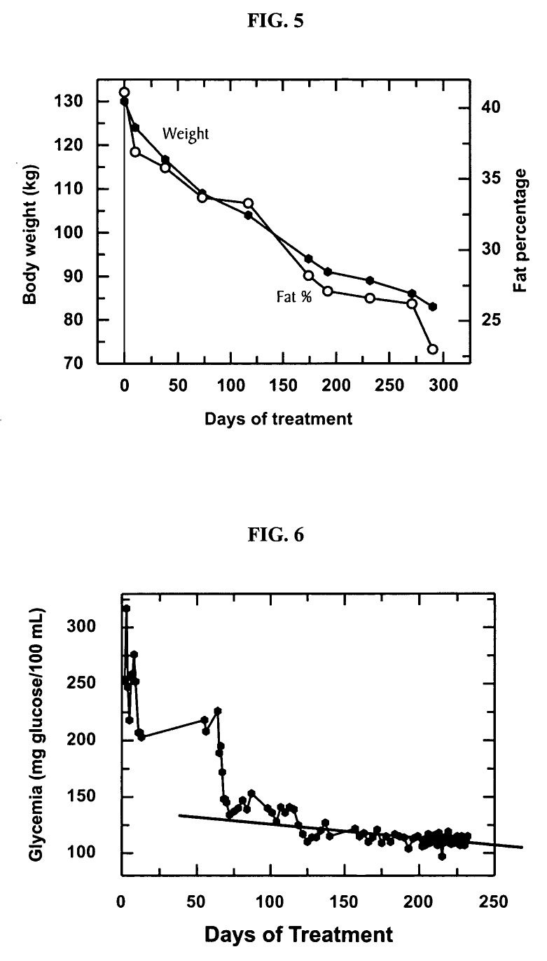

(c) Successful weight losses in all cases. Differences in the weight loss rates were due primarily to the rate at which full meals were being consumed by the patients (from 1-2 meals per day to one meal every 7-10 days). Usual weight loss rates were 5-6 kg or 11-13 pounds per month, with the rate for men often being as much as 50% greater than that for women. FIG. 5 shows the progress of a typical case.

(d) For the few patients who reported symptoms suggesting a slight carbohydrate-abstinence syndrome, we changed their diets to a more gradual elimination of carbohydrates and added one or two additional doses of L-aspartic acid. We believe that the best way to avoid this abstinence syndrome is to have L-aspartic acid with something that contains sugar, such as fruit juice or yogurt.

(e) Unlike conventional hypocaloric diets that yield degenerative weight losses, reducing muscular mass with a little fat reduction so that one's percentage body fat actually increases, our recommended treatment showed significant reductions in our patients' percentages of body fat; see Table 1.

(f) The significant fat reductions yielded by our treatments offer the opportunity for people to sculpture or alter their body shape to one that is more desirable to the patient. Examples 7, 10, 12 and 13 in Table 1 show how women who did not need significant weight losses were able to affect reductions in the size of their hips and midsections. Examples 2, 6, 8, 9 and 11 show similar results for women who did need to lose significant amounts of weight.

(g) Avoidance of baggy skin and stretch marks after significant weight loss were seen to be achieved when our recommended treatment was combined with the treatments disclosed in U.S. patent application Ser. No. 11/199,327 for the consumption of glycine.

(h) Our results (e.g., blood chemical analysis) suggest that the duration of the recommended treatments can be as long as one wants, without danger of intoxication, or until desired results are achieved (e.g., recommended % body fat: >19% for men and >29% for women). However, one may also continue taking L-aspartic acid at low doses (for instance, 2-4 grams per day) while resuming their normal three full meals per day of a compensated diet (i.e., low in starches and carbohydrates) as a means to control one's weight and percentage of body fat.

(i) The periodic blood analysis assessments of our patients (whose biochemical and hematological parameters consistently showed improvements) suggest that our recommended treatments are yielding a general improvement in the state of health of our patients.

2. Diabetes

Our clinical trails involved a group of 200 patients, 180 with type-1 and 20 with type-2. The patients were divided into four groups: (i) Type-2 diabetes patients who had been recently diagnosed and were not yet taking any oral drugs or insulin treatments, (ii) Type-2 diabetes patients who had been taking oral drugs (usually sulphonylurea derivatives) for up to several years, (iii) Type-2 diabetes patients who were suffering for many years, and had started their treatment with oral drugs and, after several years, progressed to insulin injections, which were eventually being supplemented with oral drugs, and (iv) Type-1 diabetes patients, all of them under insulin treatment. Most of these patients were also suffering from other related health problems (e.g., overweight or obesity, hypertension, sexual potency problems).

The basic treatment guidelines for these diabetes patients were essentially the same as that which was indicated above for our obesity and overweight patients (i.e., four daily takings of 1-3 grams of L-aspartic acid and the elimination of starches from one's diet).

A primary aim of this treatment program was to allow the type-2 diabetes patients to eliminate their oral drugs and/or insulin injections for, and to allow type-1 diabetes patients to reduce their insulin injections to a minimum amount. This was achieved to such a degree that patients following our treatment program had to be attentive to quickly and substantially reducing their diabetes drug consumptions in order to avoid hypoglycemia episodes.

The results of these clinical trials were that all of our diabetes patients were able to reduce to some degree their oral drugs and/or insulin injections. Most of the type-2 patients who were receiving insulin injections were able to change to oral diabetes drugs 1-2 months after starting with our treatment, and then they gradually reduced their oral drug intakes to zero. For the majority of patients who had diabetes-related health conditions, improvements were seen in these conditions. Drugs taken for hypertension were reduced or eliminated, weight reductions were achieved, and the patients almost uniformly reported that they had achieved a general improvement in their sense of well-being and their overall health.

Table 2 presents a summary of the clinical results achieved for a representative number of our patients.

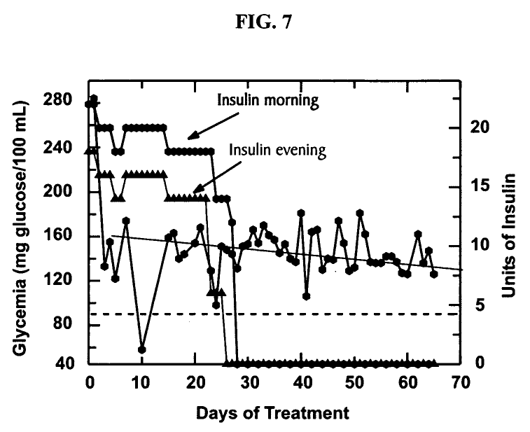

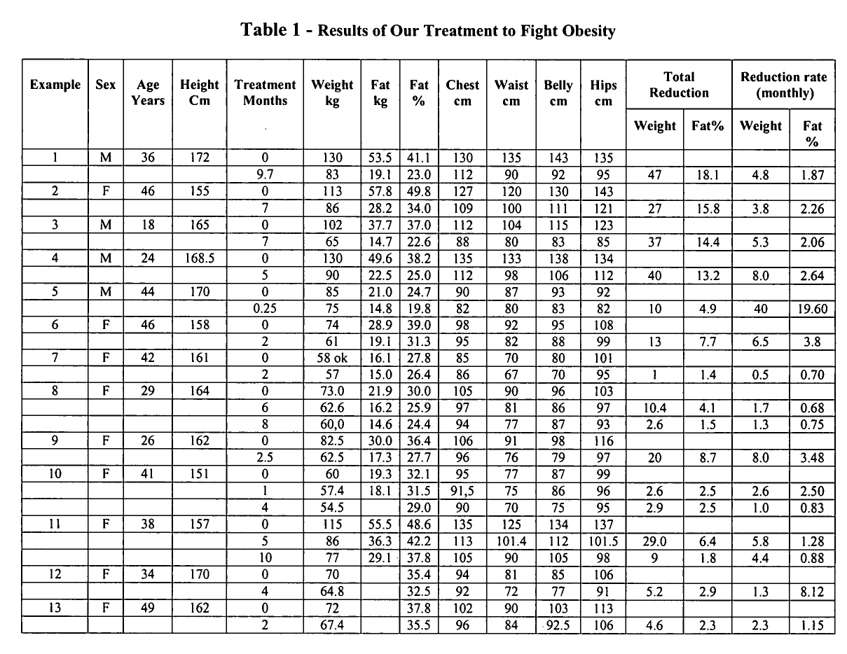

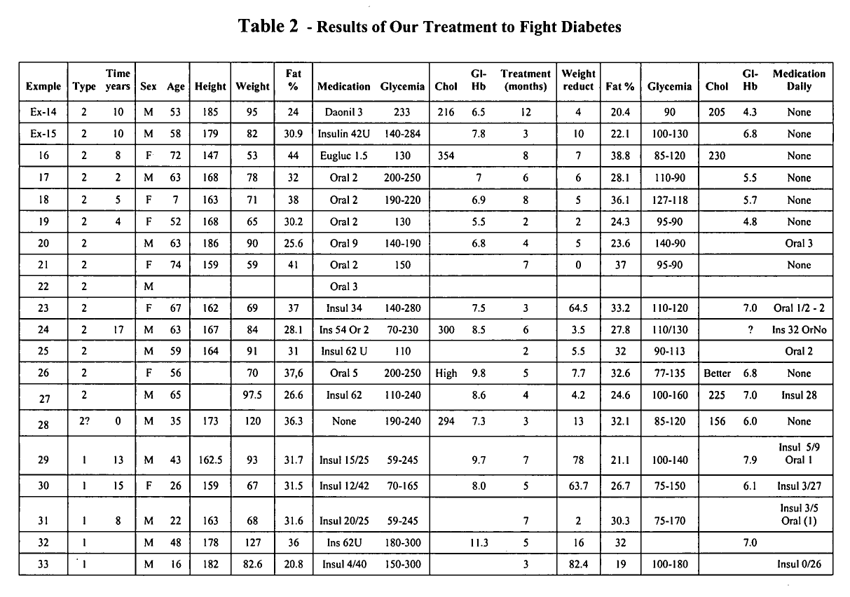

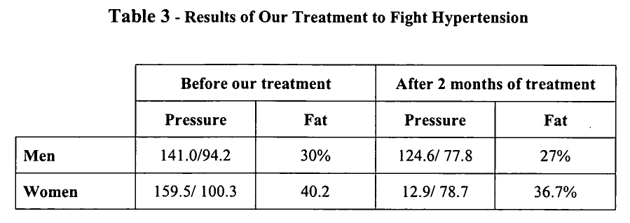

FIGS. 6 and 7 illustrate the improvements that our treatment yielded in the glycemia levels of two type-2 diabetes patients who had both been suffering from diabetes for over 10 years. See FIG. 6: Example 14 from Table 2, 53 year-old man, 95 kg, 24% fat. See FIG. 7: Example 15 from Table 2, 58 year-old man, 82 kg, 31% fat). Improvements in glycemia levels were clear from the first week of treatment. After 1-2 months, both patients achieved good control of their significantly reduced glycemia levels and were able to discontinue their diabetes medications.

Patients' chemical blood analysis showed significant improvements over the duration of their treatments. Glycosylated haemoglobin, cholesterol, tryglycerides, and total lipids, that are usually very high, were drastically reduced. See Table 2. For example, reductions of 20% and more were measured in patients' glycosylated haemoglobin levels (e.g., Example 32 in Table 2, who had a 38.05% reduction).

Improvements in other health conditions related to diabetes (e.g., gradual loss of vision, deterioration of kidney function, sexual impotency) were also reported.

While not being able to achieve the total discontinuance of insulation injections that was achieved by type-2 patients, type-i diabetes patients were able to achieve significant reductions (20-30%) in their insulin dosages over periods of 3-12 months.

3. Hypertension

Clinical trials for 250 patients suffering from hypertension (156 men and 94 women, with ages between 19 and 70 years) are reported below. Patients tested their blood pressure several times per day and gradually decreased their intake of hypertension drugs as their blood pressure decreased.

A summary of our clinical results are shown in Table 3 for average daily L-aspartic acid consumptions of 12 g/day (four daily 3 g doses). Attainment of recommended normal blood pressures was achieved in a mean time of 2.8 months, along with reductions in % body fat of approximately 10%. This result supports our hypothesis that the primary cause of high blood pressure is fat which is stored in and reduces the permeability of one's capillary walls.

An interesting result observation of thee trails was that these blood pressure reductions were often achieved while the patient's body fat percentage was still high. Our interpretation of this result is that the body consumes first its microscopic fat, which is the primary cause of hypertension, and then it consumes its macroscopic fat, which itself evident in one's degree of obesity.

4. Atherosclerosis

We have not yet had the opportunity to note the effects of our recommended treatment methods on patients suffering from atherosclerosis.

5. Migraine, Headaches and Menstrual Pains

Our treatments achieved a significant reduction in the number of patients' reporting migraines, headaches, and menstrual pains. Many patients who suffered these problems noticed a clear improvement in these conditions in the first or the second month after starting treatment. In some cases, longer durations of treatments (several months) were required.

6. Cholesterol Excess

These clinical trials included a group of more than 200 non-diabetic patients with blood cholesterol excesses. The basic treatment (i.e., four daily takings of 1-3 grams of L-aspartic acid and the elimination of starches from one's diet) for these patients was the same as that described above, plus: (i) complete elimination of carbohydrates from the diet, (ii) inclusion of unsaturated and polyunsaturated fat in all meals, and (iii) inclusion in some meals of cholesterol.

All patients achieved a clear improvement as measured by decreasing blood cholesterol, tryglycerides and total lipid levels, and all of them were eventually able to cease taking their drugs for cholesterol excesses. Contrary to the cases of high blood pressure, which were usually improved quickly, even before that body fat percentage were substantially decreased, the decrease of blood cholesterol was slow in many cases, always apparently dependent on body fat decreases.

In a few cases and during the first two to three months, patients showed a higher than initial blood cholesterol and triglyceride levels. We believe these situations to be a consequence of the excess release of cholesterol from cell membranes as it is replaced by unsaturated fat. We do not believe these increases to be dangerous as the really ‘bad’ cholesterol is the cholesterol deposited on the blood vessels, and not that which is circulating in one's blood. In the next months, the blood cholesterol levels for these patients decreased to normal values which were much lower than the patients' levels before they started the treatment.

7. Multiple Sclerosis and Alzheimer's Disease

We have not yet had the opportunity to note the effects of our recommended treatment methods on patients suffering from these disorders.

8. Anemia

We have not yet had the opportunity to note the effects of our recommended treatment methods on patients suffering from anemia.

9. High Uric Acid Levels

Sixty patients participating in this program had high blood uric acid levels. This problem showed to be much more frequent in men (48 cases) than women (12).

Our treatment achieved a significant decrease in the uric acid levels of these patients: from initial levels of between 7.1 and 9.4 mg/100 mL to, after usually 2-4 months treatment, normal values. These results suggest that the origin of this uric acid is not exogen (from meat, fish or seafood in the diet) but endogen (coming from muscle degradation).

These improvements in uric acid levels were often also associated with patients' reports of the elimination of pains in feet and the sensations of stinging skin. Some patients, once they reached low uric acid levels, decided to return to a high-carbohydrate diet and to stop the taking of L-aspartic acid just to see what would happen. The result was that, after about three months, their uric acid levels rose again and their high uric acid symptoms reappeared. Upon returning to our treatment, their uric acid levels again decreased and their symptoms.

10. Ketosis

None of the patients under our treatment have suffered any kind of ketosis. Those who exhibited signs of ketosis before starting our treatment achieved normal blood concentration of ketone bodies in a relatively short time after beginning treatments. This result was contrary to situation that is often observed when carbohydrates are restricted in diet and signs of ketosis do appear (as has been reported in those following an Atkins' diet).

11. Tobacco and Other Drug Addictions

Most patients in our trials who were addicted to tobacco and expressed an interest in ceasing smoking reported that, after 2-4 weeks of our treatments, they were able to reduce their daily numbers of cigarettes smoked—a result which many of them attributed to their observed levels of lower anxiety. Smoking patients who expressed no desire to stop also reported reductions in their levels of smoking.

Smoking patients who expressed a desire to stop were advised to prepare a small bottle of water (or even fruit juice) with some small spoonfuls of L-aspartic acid, and to drink some small swigs of this throughout the day to control their anxiety levels. After several weeks, all of these patients had ceased smoking.

Similar cessation results were achieved for caffeine addiction (patients consuming 8-12 daily coffees or soft drinks with caffeine). Our treatment was able to drastically eliminate this dependence in only a few weeks.

Some patients in our clinical trials, who had for years been taking various drugs to fight depression or anxiety, reported that they were able to eliminate or reduce the amounts of these drugs they were taking after only a few weeks of our treatment.

12. “Pregnancy Diabetes” and Fetus Development

We have not yet had the opportunity to note the effects of our recommended treatment methods on expectant mothers.

13. Cancer

We have not yet had the opportunity to note the effects of our recommended treatment methods on cancer patients.

14. Sexual Potency and Fertility

A number of the male patients in our trails initially complained of erectile disfunction and other sexual performance problems. After 2-3 months of treatment, all of these patients reported significant diminishments of their problems in this area. These improvements were most significant for those patients who had reported problems of erectile disfunction; with these patients generally reporting improvements in all aspects of their sexual activities, including their sexual appetency and ability to ejaculate.

15. Digestion Problems

More than three hundred patients who participated in our clinical trials had some kind of digestion problems including indigestion, acidic stomach, stomach bloating, stomach and intestinal gas, flatulence, bad breath (halitosis), hiatus hernia, vomiting, diarrhea and constipation. After two or three weeks of our recommended programs, all of these patients reported an almost total resolution of their digestion problems. Many of these patients who had been taking drug treatments for these problems were able to discontinue such drug treatments in only a few weeks.

An additional problem in number of these patients was an insufficient secretion of stomach proteases (and eventually intestinal proteases). This problem was greatly improved by having these patients supplement our basic treatment by consuming capsules of digestive enzymes (pepsin, papain, bromelain, pancreatin, and others) and fruits rich in proteases, such as papaya, pineapple and figs.

These results and our theories suggest that an excess of hydrochloride acid secretion is not a cause of digestion problems, but a consequence of poor digestive enzyme activity.

In concluding this disclosure, it should be noted that the foregoing is considered as illustrative only of the principles of the present invention. Further, since numerous modifications and changes will readily occur to those skilled in the art, it is not desired to limit the invention to the exact methods described herein, and accordingly, all suitable modifications and equivalents may be resorted to which fall within the scope of the invention as hereinafter forth in the claims to it.

We claim:

1. A method for treating or preventing a health problem that is associated with one's cells having a poor metabolic capability to process fat, said method comprising the steps of:
administering a therapeutically effective dosage of an anaplerotic precursor.

2. The method of claim 1, further comprising the steps of:
restricting one's consumption of starchy foods.

3. The method of claim 1, wherein said anaplerotic precursor is a glycogenic amino acid.

4. The method of claim 2, wherein said anaplerotic precursor is a glycogenic amino acid.

5. The method of claim 3, wherein said glycogenic amino acid is chosen from the group of L-aspartic acid or a pharmaceutically acceptable analog thereof.

6. The method of claim 4, wherein said glycogenic amino acid is chosen from the group of L-aspartic acid or a pharmaceutically acceptable analog thereof.

7. The method of claim 5, wherein said administration is at a prescribed number of intervals throughout the day, wherein said number of intervals is chosen so as to promote one's cellular ability to uniformly process fat throughout the day.

8. The method of claim 6, wherein said administration is at a prescribed number of intervals throughout the day, wherein said number of intervals is chosen so as to promote one's cellular ability to uniformly process fat throughout the day.

9. The method of claim 5, wherein said effective dosage is within the range of about 2-20 g/day.

10. The method of claim 6, wherein said effective dosage is within the range of about 2-20 g/day.

11. The method of claim 9, wherein said effective dosage is within the range of about 6-12 g/day.

12. The method of claim 10, wherein said effective dosage is within the range of about 6-12 g/day.

13. The method of claim 1, wherein said health problem is chosen from the group consisting of obesity and the related health conditions that are aggravated by obesity, diabetes, hypertension, atherosclerosis, migraines and headaches, menstrual cramps, cholesterol excesses, multiple sclerosis and Alzheimer's disease, anemia, high blood uric acid levels, ketosis, tobacco and other drug addictions, and digestive problems.

14. The method of claim 2, wherein said health problem is chosen from the group consisting of obesity and the related health conditions that are aggravated by obesity, diabetes, hypertension, atherosclerosis, migraines and headaches, menstrual cramps, cholesterol excesses, multiple sclerosis and Alzheimer's disease, anemia, high blood uric acid levels, ketosis, tobacco and other drug addictions, and digestive problems.

15. The method of claim 9, wherein said health problem is chosen from the group consisting of obesity and the related health conditions that are aggravated by obesity, diabetes, hypertension, atherosclerosis, migraines and headaches, menstrual cramps, cholesterol excesses, multiple sclerosis and Alzheimer's disease, anemia, high blood uric acid levels, ketosis, tobacco and other drug addictions, and digestive problems.

16. The method of claim 10, wherein said health problem is chosen from the group consisting of obesity and the related health conditions that are aggravated by obesity, diabetes, hypertension, atherosclerosis, migraines and headaches, menstrual cramps, cholesterol excesses, multiple sclerosis and Alzheimer's disease, anemia, high blood uric acid levels, ketosis, tobacco and other drug addictions, and digestive problems.

17. A compound for treating or preventing in one a health problem that is associated with one's cells having a poor metabolic capability to process fat, said compound comprising:
a therapeutically effective dosage of an anaplerotic precursor.

18. The compound of claim 17, wherein said anaplerotic precursor is a glycogenic amino acid.

19. The compound of claim 18, wherein said glycogenic amino acid is chosen from the group of L-aspartic acid or a pharmaceutically acceptable analog thereof.

20. The compound of claim 19, wherein said effective dosage is within the range of about 2-20 g/day.

21. The compound of claim 20, wherein said health problem is chosen from the group consisting of obesity and the related health conditions that are aggravated by obesity, diabetes, hypertension, atherosclerosis, migraines and headaches, menstrual cramps, cholesterol excesses, multiple sclerosis and Alzheimer's disease, anemia, high blood uric acid levels, ketosis, tobacco and other drug addictions, and digestive problems.

22. A process of improving an aspect of the well being of a subject, wherein said aspect is related to the metabolic capability of said subject's cells to process fat, said process comprising the steps of:
administering a therapeutically effective dosage of an anaplerotic precursor.

23. The process of claim 22, further comprising the steps of:
restricting the consumption of carbohydrates by said subject, except for those found in fruits, vegetables and dairy products.

24. The process of claim 23, wherein said anaplerotic precursor is a glycogenic amino acid.

25. The process of claim 24, wherein said glycogenic amino acid is chosen from the group of L-aspartic acid or a pharmaceutically acceptable analog thereof.

26. The process of claim 25, wherein said administration is at a prescribed number of intervals that is set so as to promote one's cellular ability to uniformly process fat throughout the day.

27. The process of claim 26, wherein said effective dosage is within the range of about 2-20 g/day.

28. The process of claim 27, wherein said aspect of said subject's well being which is to be improved upon is chosen from the group consisting of obesity and the related health conditions that are aggravated by obesity, diabetes, hypertension, atherosclerosis, migraines and headaches, menstrual cramps, cholesterol excesses, multiple sclerosis and Alzheimer's disease, anemia, high blood uric acid levels, ketosis, tobacco and other drug addictions, and digestive problems.

29. The use of a compound for the manufacture of a medicament treatment for one having a health problem that is associated with one's cells having a poor metabolic capability to process fat, said compound comprising:
a therapeutically effective dosage of an anaplerotic precursor.

30. The compound of claim 29, wherein said anaplerotic precursor is a glycogenic amino acid.

31. The compound of claim 30, wherein said glycogenic amino acid is chosen from the group of L-aspartic acid or a pharmaceutically acceptable analog thereof.

32. The compound of claim 31, wherein said effective dosage is within the range of about 2-20 g/day.

33. The compound of claim 32, wherein said health problem is chosen from the group consisting of obesity and the related health conditions that are aggravated by obesity, diabetes, hypertension, atherosclerosis, migraines and headaches, menstrual cramps, cholesterol excesses, multiple sclerosis and Alzheimer's disease, anemia, high blood uric acid levels, ketosis, tobacco and other drug addictions, and digestive problems.
